(19) United States
(12) Patent Application Publication
(10) Pub. No.: US 2022/0347062 A1
(43) Pub. Date: Nov. 3, 2022

(54) SUNSCREEN SPRAY

(71) Applicant: L'OREAL, Paris (FR)

(72) Inventor: Anil SHAH, East Windsor, NJ (US)

(73) Assignee: L'OREAL, Paris (FR)

(21) Appl. No.: 17/244,111

(22) Filed: Apr. 29, 2021

(51) Int. Cl. A61K 8/04; A61K 8/06; A61Q 17/04; A61K 8/86; A61K 8/60; A61K 8/55; A61K 8/37

(52) U.S. Cl. CPC A61K 8/046; A61K 8/064; A61Q 17/04; A61K 8/86; A61K 8/608; A61K 8/553; A61K 8/375; A61K 2800/34

(57) ABSTRACT

A cosmetic composition comprising an aqueous phase; at least one UV filter; an oil phase; an emulsifier system comprising from about 0.05 to about 1 wt. % of one ionic surfactant; from about 2.5 to 4.0 wt. % of one non-ionic surfactant with an HLB greater than or equal to about 14; from about 0.5 to 1.5 wt. % of one non-ionic surfactant with an HLB around about 10; from about 0.25 to 0.75 wt. % of one non-ionic surfactant with an HLB less than or equal to about 6; and from about 0.25 to 0.75 wt. % of one anionic surfactant, a water/oil phase ratio which is from about 1.3:1.0 to about 2.0:1.0 wt. %; and wherein the cosmetic composition is an emulsion with a viscosity less than 40.0 UD (spindle M1); and wherein the emulsion produces a mist spray without requiring to be mixed with propellant.

### FIELD OF THE DISCLOSURE

The instant disclosure is directed to sunscreen compositions, and to methods for using the sunscreen compositions to protect keratinous substrates such as skin and hair from UV radiation.

### BACKGROUND OF THE DISCLOSURE

Conventional sunscreen products generally contain ultraviolet (UV)-filter compounds and/or particulate UV-screening compounds (collectively, “sunscreen actives”) that are solubilized, emulsified, or dispersed in a vehicle, which is topically applied to the skin. The sunscreen actives, typically through the aid of polymers and other ingredients included in the vehicle form a thin, protective, and water-resistant layer on the skin.

There are many sun care products in the market in the forms of spray, lotion and cream. Sunscreen spray products are popular due to ease of application and pleasant aesthetics. The spray products in the market are either alcoholic sprays or emulsion-based lotion sprays, and often require the use of a propellant to effectuate the breakup of the formula into fine droplets. Alcoholic sprays provide a mist spray mixed with propellant and without propellant, but the drawbacks of alcoholic sprays are inflammability and making the skin dry. Emulsion-based spray sun care formulations can be used to avoid the drawbacks of alcoholic spray formulations; however, there is a challenge to control ingredients and viscosity in emulsions to allow for the creation of a fine mist spray, fluidity, and stability. Creation of a fine mist spray using an emulsion base is especially challenging without the mixing with propellant.

It is thus an object of the instant disclosure to provide a sunscreen product that is an emulsion-based spray which is stable, fluid like water and provides a very fine mist spray without being mixed with propellant.

### BRIEF SUMMARY OF THE DISCLOSURE

The instant disclosure relates to a cosmetic composition that provides an emulsion-based spray which is fluid like water and provides very fine mist spray without being mixed with propellant.

The cosmetic compositions typically include:
a. an aqueous phase;
b. at least one UV filter;
c. an oil phase;
d. an emulsifier system comprising:
   i. from about 1.0 to 5.0 wt. % of one non-ionic surfactant with an HLB greater than or equal to about 14;
   ii. from about 0.5 to 1.5 wt. % of one non-ionic surfactant with an HLB around about 10;
   iii. from about 0.25 to 0.75 wt. % of one non-ionic surfactant with an HLB less than or equal to about 6; and
   iv. from about 0.25 to 0.75 wt. % of one anionic surfactant,
e. a water/oil phase ratio which is from about 1.3:1 to about 2.0:1.0; and
   wherein the cosmetic composition is an emulsion with a viscosity less than about 40.0 UD, M1 spindle; and
   wherein all percentages by weight are based on the total weight of the cosmetic composition.

In one or more embodiments, the composition is fluid like water. In some embodiments, the composition is free of silicones.

In one or more embodiments, the oil phase is present in an amount from about 1 to about 15% by weight based on the total weight of the cosmetic composition.

In one or more embodiments, the cosmetic composition is an emulsion that produces a mist spray without requiring mixture with propellant.

In some embodiments, the emulsifier system is present in an amount from about 2 to about 7% by weight based on the total weight of the cosmetic composition.

In one or more embodiments, the anionic surfactant is chosen from alkali metal salts of dicetyl phosphate and of dimyristyl phosphate; alkali metal salts of cholesterol sulphate; alkali metal salts of cholesterol phosphate; and mixtures thereof.

In one or more embodiments, the non-ionic surfactant with an HLB greater than or equal to about 14 is chosen from esters of polyethylene glycol and stearic acid—PEG-20 Stearate, PEG-32 Stearate, PEG-40 Stearate, PEG-50 Stearate, PEG-100 Stearate, PEG-150 Stearate and mixtures thereof.

In some embodiments, the non-ionic surfactant with an HLB around about 10 is chosen from polyglycerides, polysorbate 61, polysorbate 65, polysorbate 81, polysorbate 85, polyglyceryl-2 laurate, and mixtures thereof.

In one or more embodiments, the non-ionic surfactant with HLB less than or equal to about 6 is chosen from sucrose distearate, sorbitan stearate, octyldodecyl xyloside, glyceryl stearate, and mixtures thereof.

In some embodiments, the cosmetic composition has an SPF in a range from about 15 to about 55. In one or more embodiments, the cosmetic composition has an SPF in a range from about 55 to about 100. In some embodiments, the at least one UV filter is chosen from an organic UV filter and/or a mineral UV filter.

In some embodiments, the water/oil phase ratio is from about 1.3:1.0 to 2.0:1.0.

In some embodiment, the cosmetic compositions may comprise:
a. an aqueous phase;
b. at least one UV filter;
c. an oil phase;
d. an emulsifier system comprising:
   i. from about 2.5 to 4.0 wt. % of one non-ionic surfactant with an HLB greater than or equal to about 14 is chosen from esters of polyethylene glycol and stearic acid—PEG-20 Stearate, PEG-32 Stearate, PEG-40 Stearate, PEG-50 Stearate, PEG-100 Stearate, PEG-150 Stearate and mixtures thereof;
   ii. from about 0.5 to 1.5 wt. % of one non-ionic surfactant with an HLB around about 10 is chosen from polyglycerides, polysorbate 61, polysorbate 65, polysorbate 81, polysorbate 85, polyglyceryl-2 laurate, and mixtures thereof;
   iii. from about 0.25 to 0.75 wt. % of one non-ionic surfactant with an HLB less than or equal to about 6 is chosen from sucrose distearate, sorbitan stearate, octyldodecyl xyloside, glyceryl stearate, and mixtures thereof; and
   iv. from about 0.25 to 0.75 wt. % of one anionic surfactant is chosen from alkali metal salts of dicetyl phosphate and of dimyristyl phosphate; alkali metal salts of cholesterol sulphate; alkali metal salts of cholesterol phosphate; and mixtures thereof.
e. a water/oil phase ratio which is from about 1.3:1.0 to about 2.0:1.0; and
   wherein the cosmetic composition is an emulsion with a viscosity less than about 40.0 UD, M1 spindle; and
   wherein the emulsion produces a mist spray without requiring mixture with a propellant; and
   wherein all percentages by weight are based on the total weight of the cosmetic composition.

The instant disclosure is also directed to a method of inhibiting UV radiation from contacting a keratinous substrate by applying the above disclosed cosmetic composition onto a surface of the keratinous substrate.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

As used herein, the expression “at least one” means one or more and thus includes individual components as well as mixtures/combinations.

“Cosmetically acceptable” means compatible with any keratinous substrate. For example, “cosmetically acceptable carrier” means a carrier that is compatible with any keratinous substrate.

A “physiologically acceptable medium” means a medium which is not toxic and can be applied to the skin, lips, hair, scalp, lashes, brows, nails or any other cutaneous region of the body. The composition of the instant disclosure may especially constitute a cosmetic or dermatological composition.

The terms “fluid like water” as used herein means the formula is a pseudo-Newtonian fluid or that viscosity is largely independent of shear rate in addition to having the aforementioned low viscosity.

The instant disclosure relates to an emulsion-based cosmetic composition which is surprisingly fluid like water and provides a very fine mist spray without requiring being mixed with propellant.

The cosmetic compositions typically include:
a. an aqueous phase;
b. at least one UV filter;
c. an oil phase;
d. an emulsifier system comprising:
   i. from about 1.0 to 5.0 wt. % of one non-ionic surfactant with an HLB greater than or equal to about 14;
   ii. from about 0.5 to 1.5 wt. % of one non-ionic surfactant with an HLB around about 10;
   iii. from about 0.25 to 0.75 wt. % of one non-ionic surfactant with an HLB less than or equal to about 6; and
   iv. from about 0.25 to 0.75 wt. % of one anionic surfactant,
e. a water/oil phase ratio which is from about 1.3:1.0 to about 2.0:1.0; and
   wherein the cosmetic composition is an emulsion with a viscosity less than about 40.0 UD, M1 spindle; and
   wherein all percentages by weight are based on the total weight of the cosmetic composition.

### Emulsifier System

The emulsifier system is comprised of at least one nonionic surfactant having an HLB of greater than or equal to about 14, at least one nonionic surfactant having an HLB around about 10, at least one non-ionic surfactant having an HLB of less than or equal to about 6 and at least one anionic surfactant.

Suitable nonionic surfactants having an HLB greater than or equal to about 14 include, but are not limited to esters of polyethylene glycol and stearic acid—PEG-20 Stearate, PEG-32 Stearate, PEG-40 Stearate, PEG-50 Stearate, PEG-100 Stearate, PEG-150 Stearate and mixtures thereof.

A particularly preferred emulsifier is PEG-40 stearate.

The at least one nonionic surfactant having an HLB of greater than or equal to about 14 will typically be employed in an amount of from about 1.0 to 5.0% by weight, preferably from about 1.25 to 4.0% by weight, and most preferably from about 2.0 to 3.5% by weight, based on the total weight of the composition.

Suitable nonionic surfactants having an HLB of around about 10 include, but are not limited to, polyglycerides, polysorbate 61, polysorbate 65, polysorbate 81, polysorbate 85, and mixtures thereof.

Particularly preferred nonionic surfactants having an HLB of around about 10 include polysorbate 61 and polyglyceryl-2 laurate.

The at least one nonionic surfactant having an HLB of around about 10 will typically be employed in an amount of from about 0.25% to 3% by weight, preferably from about 0.35 to 2% by weight, and most preferably from about 0.5 to 1.5% by weight, based on the total weight of the composition.

Suitable nonionic surfactants having an HLB of less than or equal to about 6 include, but are not limited to sucrose distearate, sorbitan stearate, octyldodecyl xyloside, glyceryl stearate, and combinations thereof.

The at least one nonionic surfactant having an HLB of less than or equal to about 6 will typically be employed in an amount of from about 0.1% to 2% by weight, preferably from about 0.2 to 1.0% by weight, and most preferably from about 0.25 to 0.5% by weight, based on the total weight of the composition.

Suitable anionic surfactants for use in the instant disclosure include, but are not limited to:
alkali metal salts of dicetyl phosphate and of dimyristyl phosphate;
alkali metal salts of cholesterol sulphate;
alkali metal salts of cholesterol phosphate;

Particularly preferred ionic surfactant is Potassium Cetyl Phosphate.

The ionic surfactant will typically be employed in an amount of from about 0.05 to about 1.0% by weight, preferably from about 0.1 to about 0.75% by weight, and most preferably from about 0.2 to about 0.5% by weight, based on the total weight of the composition.

The emulsifier system will typically be present in the composition in an amount of up to about 7% by weight, preferably up to about 5.5% by weight, and most preferably up to about 5.1% by weight, based on the total weight of the composition.

It is imperative that the emulsifier system be employed in the above-disclosed amounts.

### Oil Phase

The oil phase of the instant disclosure is comprised of at least one oil.

Suitable oils include, but are not limited to;
mineral oils, such as hexadecane, isohexadecane and liquid paraffin;
animal or plant oils formed by fatty acid esters of polyols, in particular liquid triglycerides, for example sunflower oil, corn oil, soybean oil, avocado oil, jojoba oil, marrow oil, grapeseed oil, sesame oil, hazelnut oil, fish oils, glyceryl tricaprocaprylate, purcellin oil, or liquid jojoba wax;
natural or synthetic essential oils such as, for example, eucalyptus oil, lavandin oil, lavender oil, vetivier oil, litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;
synthetic oils such as parleam oil, polyolefins and liquid carboxylic acid esters;
halogenated oils, in particular fluorocarbons, such as fluoroamines, for example perfluorotributylamine, fluorinated hydrocarbons, for example perfluoro-decahydronaphthalene, fluoroesters and fluoroethers;
volatile and non-volatile silicone oils;
polyolefins, in particular poly-α-olefins, and more particularly those of hydrogenated or nonhydrogenated polybutene type, and preferably hydrogenated or nonhydrogenated polyisobutene type;
esters of mono-, di-, tri- or tetracarboxylic acids, in particular alkyl palmitates, such as ethyl palmitate, isopropyl palmitate, 2-ethylhexyl palmitate, 2-octyldecyl palmitate; alkyl myristates, such as isopropyl myristate, butyl myristate, cetyl myristate, 2-octyldodecyl myristate; alkyl stearates, such as hexyl stearate, butyl stearate or isobutyl stearate; alkyl malates, such as dioctyl malate; alkyl laurates, such as hexyl laurate and 2-hexyldecyl laurate, isononyl isononanoate, or cetyl octanoate; and mixtures thereof.

The oil phase will typically be employed in an amount of from about 1 to 15% by weight, preferably from about 2 to 10% by weight, and more preferably from about 3% to 8% by weight, based on the total weight of the composition.

### Water/Oil Ratio

The weight ratio of the total amount of water to the total amount of oil phase is typically about 1.3:1.0 to about 2.0:1.0.

In some instances, the ratio of the total amount of water to the total amount of oil phase is 1.3:1.0 to 1.9:1.0, 1.3:1.0 to 1.8:1.0, 1.3:1.0 to 1.7:1.0, to 1.4:1.0 to 2.0:1.0, 1.4:1.0 to 1.9:1.0, 1.4:1.0 to 1.8:1.0, to 1.4:1.0 to 1.7:1.0 or 1.4:1.0 to 1.6:1.0.

### Sunscreen Actives

Suitable UV-screening agents include, but are not limited to, cinnamic derivatives; anthranilates; salicylic derivatives; dibenzoylmethane derivatives; camphor derivatives; benzophenone derivatives; β,β-diphenylacrylate derivatives; triazine derivatives; benzotriazole derivatives; benzalmalonate derivatives, especially those cited in U.S. Pat. No. 5,624,663; benzimidazole derivatives; imidazolines; bis-benzoazolyl derivatives as described in patents EP669323 and U.S. Pat. No. 2,463,264; p-aminobenzoic acid (PABA) derivatives; methylene bis(hydroxyphenylbenzotriazole) derivatives as described in applications U.S. Pat. Nos. 5,237,071, 5,166,355, GB2303549, DE19726184 and EP893119; benzoxazole derivatives as described in patent applications EP0832642, EP1027883, EP1300137 and DE10162844; screening polymers and screening silicones such as those described especially in patent application WO 93/04665; dimers derived from α-alkylstyrene such as those described in patent application DE 19855649; 4,4-diarylbutadienes such as those described in patent applications EP0967200, DE19746654, DE19755649, EP-A-1008586, EP1133980 and EP1133981, merocyanine derivatives such as those described in patent applications WO 04/006878, WO 05/058269 and WO 06/032741; and mixtures thereof.

As examples of complementary organic photoprotective agents, mention may be made of those denoted herein below under their INCI name:

### Cinnamic Derivatives:

Ethylhexyl Methoxycinnamate sold in particular under the trade name “Parsol® MCX” by DSM Nutritional Products, Isopropyl Methoxycinnamate, Isoamyl Methoxycinnamate sold under the trade name “Neo Heliopan® E 1000” by Symrise, DEA Methoxycinnamate, Diisopropyl Methylcinnamate, Glyceryl Ethylhexanoate Dimethoxycinnamate.

### Dibenzoylmethane Derivatives:

Butyl Methoxydibenzoylmethane sold especially under the trade name “Parsol® 1789” by DSM, Isopropyl Dibenzoylmethane.

### Para-Aminobenzoic Acid Derivatives:

PABA, Ethyl PABA, Ethyl Dihydroxypropyl PABA, Ethylhexyl dimethyl PABA sold in particular under the name “Escalol™ 507” by ISP, Glyceryl PABA, PEG-25 PABA sold under the name “Uvinul® P25” by BASF.

### Salicylic Derivatives:

Homosalate sold under the name “Eusolex® HMS” by Rona/EM Industries, Ethylhexyl Salicylate sold under the name “Neo Heliopan® OS” by Symrise, Dipropylene Glycol Salicylate sold under the name “Dipsal™” by Scher, TEA Salicylate sold under the name “Neo Heliopan® TS” by Symrise.

### β,β-Diphenylacrylate Derivatives:

Octocrylene sold in particular under the trade name “Uvinul® N539” by BASF, Etocrylene sold in particular under the trade name “Uvinul® N35” by BASF.

### Benzophenone Derivatives:

Benzophenone-1 sold under the trade name “Uvinul® 400” by BASF, Benzophenone-2 sold under the trade name “Uvinul® D50” by BASF, Benzophenone-3 or Oxybenzone sold under the trade name “Uvinul® M40” by BASF, Benzophenone-4 sold under the trade name “Uvinul® MS40” by BASF, Benzophenone-5, Benzophenone-6 sold under the trade name “Helisorb® 11” by Norquay, Benzophenone-8 sold under the trade name “Spectra-Sorb UV-24” by American Cyanamid, Benzophenone-9 sold under the trade name “Uvinul® DS-49” by BASF, Benzophenone-12, n-Hexyl 2-(4-diethylamino-2-hydroxybenzoyl)benzoate sold under the trade name “Uvinul® A+” or as a mixture with octyl methoxycinnamate under the trade name “Uvinul® A+B” by BASF.

### Benzylidenecamphor Derivatives:

3-Benzylidene Camphor manufactured under the name “Mexoryl™ SD” by Chimex, 4-Methylbenzylidene Camphor sold under the name “Eusolex® 6300” by Merck, Benzylidene Camphor Sulfonic Acid manufactured under the name “Mexoryl™ SL” by Chimex, Camphor Benzalkonium Methosulfate manufactured under the name “Mexoryl™ SO” by Chimex, Terephthalylidene Dicamphor Sulfonic Acid manufactured under the name “Mexoryl™ SX” by Chimex, Polyacrylamidomethyl Benzylidene Camphor manufactured under the name “Mexoryl™ SW” by Chimex.

### Phenylbenzimidazole Derivatives:

Phenylbenzimidazole Sulfonic Acid sold in particular under the trade name “Eusolex® 232” by Merck, Disodium Phenyl Dibenzimidazole Tetrasulfonate sold under the trade name “Neo Heliopan® AP” by Symrise.

### Phenylbenzotriazole Derivatives:

Drometrizole Trisiloxane sold under the name “Silatrizole” by Rhodia Chimie, Methylene bis-Benzotriazolyl Tetramethylbutyl-phenol sold in solid form under the trade name “MIXXIM BB/100” by Fairmount Chemical, or in micronized form as an aqueous dispersion under the trade name “Tinosorb M” by Ciba Specialty Chemicals.

### Triazine Derivatives:

bis-Ethylhexyloxyphenol Methoxyphenyl Triazine sold under the trade name “Tinosorb® S” by BASF, Ethylhexyl Triazone sold in particular under the trade name “Uvinul® T150” by BASF, Diethylhexyl Butamido Triazone sold under the trade name “Uvasorb® HEB” by Sigma 3V, 2,4,6-tris(dineopentyl 4′-aminobenzalmalonate)s-triazine, 2,4,6-tris(diisobutyl 4′-aminobenzalmalonate)s-triazine, 2,4-bis(dineopentyl 4′-aminobenzalmalonate)-6-(n-butyl 4′-aminobenzoate)-s-triazine, symmetrical triazine screening agents described in U.S. Pat. No. 6,225,467, patent application WO 2004/085412 (see compounds 6 and 9) or the document “Symmetrical Triazine Derivatives” IP.COM Journal, IP.COM Inc., West Henrietta, N.Y., US (20 Sep. 2004), especially 2,4,6-tris(biphenyl)-1,3,5-triazines (in particular 2,4,6-tris(biphenyl-4-yl)-1,3,5-triazine and 2,4,6-tris(terphenyl)-1,3,5-triazine, which is included in patent applications WO 06/035000, WO 06/034982, WO 06/034991, WO 06/035007, WO 2006/034992 and WO 2006/034985).

### Anthranilic Derivatives:

Menthyl Anthranilate sold under the trade name “Neo Heliopan® MA” by Symrise.

### Imidazoline Derivatives:

Ethylhexyl Dimethoxybenzylidene Dioxoimidazoline Propionate.

### Benzalmalonate Derivatives:

Polyorganosiloxane containing benzalmalonate functions, for instance Polysilicone-15, sold under the trade name “Parsol® SLX” by DSM Nutritional Products.

### 4,4-Diarylbutadiene Derivatives:

1,1-dicarboxy(2,2′-dimethylpropyl)-4,4-diphenylbutadiene.

### Benzoxazole Derivatives:

2,4-bis[5-(1-dimethylpropyl)benzoxazol-2-yl-(4-phenyl)imino]-6-(2-ethylhexyl)imino-1,3,5-triazine sold under the name Uvasorb® K2A by Sigma 3V, and mixtures thereof.
   Preferred Organic Screening Agents are Chosen from:
Ethylhexyl Methoxycinnamate, Ethylhexyl Salicylate, Homosalate, Butyl Methoxydibenzoylmethane, Octocrylene, Phenylbenzimidazole Sulfonic Acid, Benzophenone-3, Benzophenone-4, Benzophenone-5, n-Hexyl 2-(4-diethylamino-2-hydroxybenzoyl)benzoate, 4-Methylbenzylidene Camphor, Terephthalylidene Dicamphor Sulfonic Acid, Disodium Phenyl Dibenzimidazole Tetrasulfonate, Methylene Bis-Benzotriazolyl Tetramethylbutylphenol, Bis-Ethylhexyloxyphenol Methoxyphenyl Triazine, Ethylhexyl triazone, Diethylhexyl Butamido Triazone, 2,4,6-Tris(dineopentyl 4′-aminobenzalmalonate)-s-triazine, 2,4,6-Tris(diisobutyl 4′-aminobenzalmalonate)-s-triazine, 2,4-Bis(dineopentyl 4′-aminobenzalmalonate)-6-(n-butyl 4′-aminobenzoate)-s-triazine, 2,4,6-Tris(biphenyl-4-yl)-1,3,5-triazine, 2,4,6-Tris(terphenyl)-1,3,5-triazine, Drometrizole Trisiloxane, Polysilicone-15, 1,1-dicarboxy(2,2′-dimethylpropyl)-4,4-diphenylbutadiene, 2,4-bis[5-1(dimethylpropyl)benzoxazol-2-yl-(4-phenyl)imino]-6-(2-ethylhexyl)imino-1,3,5-triazine, and mixtures thereof.

The sunscreen actives according to the invention are typically present in the composition in an amount ranging from about 25 to 40% by weight, preferably from about 25% to 35% by weight, and most preferably from about 25 to 30% by weight, based on the total active weight of the composition, if a composition having an SPF of from about 50 to 100 is desired.

On the other hand, if a composition having an SPF of from about 15 to about 50 is desired, the sunscreen actives will be employed in an amount ranging from about 10 to 25% by weight, preferably from about 12% to 25% by weight, and most preferably from about 15 to 25% by weight, based on the total active weight of the composition.

### Optional Solvents

The composition may optionally comprise at least one solvent. The at least one solvent may be chosen from:
glycols, such as glycerol, propylene glycol, 1,3 butylene glycol, caprylyl glycol, dipropylene glycol or polyethylene glycols comprising from 4 to 16 ethylene oxide units, and preferably from 8 to 12;

The at least one solvent may be employed in an amount of from about 0.25 to 15% by weight, preferably from about 1 to 10% by weight, and more preferably from about 2 to 8% by weight, based on the total weight of the composition.

The composition of the invention may also contain adjuvants, and in particular water-soluble or liposoluble active agents having a cosmetic or dermatological activity. By way of examples of active agents, mention may be made of vitamins and their derivatives, such as vitamin E and its esters, for instance vitamin E acetate, vitamin C and its esters, B vitamins, vitamin A alcohol or retinol and its esters, such as vitamin A palmitate, vitamin A acid or retinoic acid and its derivatives, provitamins such as panthenol and niacinamide, ergocalciferol, antioxidants, essential oils, humectants, sunscreens, moisturizers, proteins, ceramides and pseudoceramides, and DHEA and its derivatives and biological precursors. As adjuvants, mention may also be made of sequestering agents, preserving agents, fillers, softeners, dyestuffs (pigments or dyes) and fragrances.

The amounts of these various adjuvants are those conventionally used in the field under consideration, and are, for example, from 0.01% to 20% of the total weight of the composition. These adjuvants and the concentrations thereof should be such that they do not modify the property desired for the composition of the invention.

The emulsifier system will typically be present in the composition in an amount of up to about 7% by weight, preferably up to about 5.5% by weight, and most preferably up to about 5.1% by weight, based on the total weight of the composition It is imperative that the emulsifier system be employed in the above-disclosed amounts so that the stabilization effect of the emulsifier to avoid flocculation and phase separation is balanced with the desire for fluidity and ability to spray the formula. While prior art can be used to formulate stable oil-in-water sun care emulsions, the emulsifier mixtures described within the prior art are unable to stabilize a highly fluid emulsion which forms the basis for the present invention. See U.S. Pat. No. 9,820,920. Without intending to be bound by theory, it is believed that controlling the interfacial water-oil tension via a specific mixture of emulsifiers and the water-to-oil phase ratio is responsible for the desired formula behaviors and stability.

### EXAMPLES

### Example 1

### Inventive Example

Sunscreen compositions in the form of an oil-in-water emulsion were studied. Details for the sunscreen compositions are provided in Table 1 below.

TABLE 1
Inventive Example

| | | Inventive |
|---|---|---|
| | | Example |
| Phase | INCI name | (Ex. 1) |
| A | Sunscreen Actives | 29 |
| A | Emollients | 6 |
| A | PEG-40 STEARATE | 3.1 |
| A | POLYSORBATE 61 | 1 |
| A | POTASSIUM CETYL | 0.5 |
| | PHOSPHATE | |
| A | GLYCERYL STEARATE | 0.5 |
| A | Preservatives | 1.3 |
| B | DISODIUM EDTA | 0.1 |
| B | WATER | 58.4 |
| C | TOCOPHEROL | 0.1 |

In making examples in Tables 1 and 2, the following procedure was used.
1. Dissolve raw materials of phase A at 70° C.
2. Dissolve raw materials of phase B at 70˜C.
3. Stir phase A at 500 rpm with an overhead propeller mixer and add phase B to phase A quickly, Homogenized for 20 minutes
4. Add phase C at 40˜C. and homogenized for 5 minutes.
5. Observe product under 40× microscope and if solution is homogenous, then begin cool down to 25˜C.

### Example 2

### Experimental Data

Oil-in-water sunscreens emulsions were studied in order to show that the combination of certain range of HLB with very low viscosity is important to get a stable emulsion based formula which is stable, and provides a very fine mist spray without requiring to be mixed with propellant. The results are presented in Table 2 below. The comparative examples presented in Table 2 were prepared according to the procedure described in Example 1.

TABLE 2
Viscosity and Stability data of Inventive and Comparative Examples

| | | Comp. | Comp. | Comp. | Comp. | Comp. | Comp. | Comp. |
|---|---|---|---|---|---|---|---|---|
| Emulsifier (HLB) | Ex. 1 | Ex. 1 | Ex. 2 | Ex. 3 | Ex. 4 | Ex. 5 | Ex. 6 | Ex. 7 |
| Other oily phase | 36.4 | 36.4 | 36.9 | 36.4 | 37.04 | 40.8 | 44.94 | 51.35 |
| ingredients | | | | | | | | |
| Other water phase | 58.5 | 59.1 | 58.6 | 59.25 | 58.71 | 54.95 | 50.81 | 44.15 |
| ingredients | | | | | | | | |
| POLYSORBATE 60 | 0 | 0 | 0 | 0 | 1.5 | 0 | 1.5 | 0 |
| (14.9) | | | | | | | | |
| PEG-40 STEARATE | 3.1 | 1.5 | 1.5 | 3.1 | 0 | 1.5 | 0 | 1.5 |
| (17.5) | | | | | | | | |
| DISODIUM | 0 | 0 | 0.5 | 0.25 | 0.25 | 0.25 | 0.25 | 0 |
| STEAROYL | | | | | | | | |
| GLUTAMATE (35) | | | | | | | | |
| POTASSIUM CETYL | 0.5 | 0.5 | 0 | 0 | 0 | 0 | 0 | 0.5 |
| PHOSPHATE (9) | | | | | | | | |
| POLYSORBATE 61 | 1 | 1 | 1 | 1 | 2.5 | 2.5 | 2.5 | 1 |
| (9.6) | | | | | | | | |
| GLYCERYL STEARATE | 0.5 | 0 | 0 | 0 | 0 | 0 | 0 | 0 |
| (3.8) | | | | | | | | |
| SORBITAN | 0 | 1.5 | 1.5 | 0 | 0 | 0 | 0 | 1.5 |
| TRISTEARATE (2.1) | | | | | | | | |
| Calculated HLB | 13.8 | 9.7 | 12.6 | 16.7 | 13.0 | 13.9 | 13.0 | 9.7 |
| Viscosity (M1 spindle) | 38.6 UD | N/A | N/A | N/A | 52.0 UD | 49.7 UD | 63.4 UD | 99 UD |
| Stability | STABLE | NOT | NOT | NOT | STABLE | STABLE | STABLE | STABLE |
| | | STABLE | STABLE | STABLE | | | | |
| Sprayability (fine Mist) | Yes | N/A | N/A | N/A | Not fine | Not fine | No | No |
| | | | | | mist | mist | | |
| Water/Oil Phase Ratio | 1.41 | 1.44 | 1.44 | 1.47 | 1.44 | 1.23 | 1.04 | 0.79 |

Viscosity should be lower than 40.0 UD (M1 spindle)
Formula should be stable
Inventive formula has very low viscosity (38.6 UD; M1 spindle) and stable while comparative formulas are either not stable or have a higher viscosity.

The following procedures to measure the viscosity and the stability of the Examples were used:

Stability: All the formulas were kept in oven at four different temperatures (5˜C, 25˜C., 45˜C. and 37˜C.) for 12 weeks. The formulas were observed visually for appearance, color change and any separation or sedimentation as well as examined for emulsion quality under microscope every week.
Viscosity: A Rheometer model no. RM 180 from Maple instrument was used to measure the apparent viscosity of the formulas. All measurements herein reported are in apparent viscosity (UD values), using TV spindle geometry.
Sprayability (fine Mist): Formula was filled and dispensed in a continuous spray packaging which did not mix the formula with propellant. Determination of spray quality was visually observed via multiple dispenses, each 0.5 to 5 seconds in duration. Water/Oil Phase Ratio: Calculated by dividing total ingredient wt % in the water phase by the total ingredient wt % of the oily phase. All emulsifiers in the examples are hydrophobic except Sodium Stearoyl Glutamate. All hydrophobic emulsifiers are considered in this calculation to be included in the oily phase and Sodium Stearoyl Glutamate is considered in this calculation to be included in the water phase.

### Results

According to the data collected for the inventive and comparative examples, it was apparent that the emulsifier system was critical, as well as the specific phase ratio of water and oil. Indeed, the results demonstrated that in order to be an inventive formula (i.e. fluid like water, providing a very fine mist without requiring mixture with propellant, and stable), the viscosity of the compositions needed to be very low. The inventive example exhibited a viscosity of 38.6 UD and was stable while the comparative examples were either not stable or have a higher viscosity. Comparative examples 1, 2 and 3 had a similar water/oil phase ratio but the surfactant system was different than inventive example and showed that they were not stable. Comparative example 4 had a similar water/oil phase ratio as inventive example and it was stable but due to different surfactant system the viscosity of the formula was higher and did not spray mist. Comparative example 5, 6 & 7 were stable but due to different water/oil phase ratio and different surfactant system, all three formulas had higher viscosity and didn't spray mist. All these results demonstrated that in order to get a very fluid stable emulsion which could spray fine mist, it was very critical that the viscosity of the compositions should be very low and it could be achieved with the inventive water/oil phase ratio and inventive surfactant system.

While the disclosure has been described with reference to a preferred embodiment, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one” or “one or more” or “one or a combination of” or “more than one” as used herein, means that there may be one, two, three or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the sunscreen composition, typically 0% by weight, based on the total weight of the sunscreen composition. The term “essentially free” means that, while it prefers that no excluded material is present in the sunscreen composition, it is possible to have very small amounts of the excluded material in the sunscreen composition of the invention, provided that these amounts do not materially affect the advantageous properties of the sunscreen composition. In particular, “essentially free” means that excluded material can be present in the sunscreen composition at an amount of less than about 0.1% by weight, based on the total weight of the sunscreen composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A cosmetic composition comprising:
a. an aqueous phase;
b. at least one UV filter;
c. an oil phase;
d. an emulsifier system comprising:
i. from about 1.0 to 5.0 wt. % of one non-ionic surfactant with an HLB greater than or equal to about 14;
ii. from about 0.25 to 3.0 wt. % of one non-ionic surfactant with an HLB around about 10;
iii. from about 0.25 to 0.75 wt. % of one non-ionic surfactant with an HLB less than or equal to about 6; and
iv. from about 0.25 to 0.75 wt. % of one anionic surfactant,
e. a water/oil phase ratio which is from about 1.3:1.0 to about 2.0:1.0; and
wherein the cosmetic composition is an emulsion with a viscosity less than about 40.0 UD (spindle M1); and
wherein all percentages by weight are based on the total weight of the cosmetic composition.

2. The cosmetic composition according to claim 1, wherein the composition is fluid like water.

3. The cosmetic composition according to claim 1, wherein the composition is free of silicones.

4. The cosmetic composition according to claim 1, wherein the emulsion produces a mist spray without requiring mixture with propellant.

5. The cosmetic composition according to claim 1, wherein the oil phase is present in an amount from about 1 to about 15% by weight base on the total weight of the cosmetic composition.

6. The cosmetic composition according to claim 1, wherein the emulsifier system is present in an amount from about 2 to about 7% by weight base on the total weight of the cosmetic composition.

7. The cosmetic composition according to claim 1, wherein the anionic surfactant is chosen from alkali metal salts of dicetyl phosphate and of dimyristyl phosphate; alkali metal salts of cholesterol sulphate; alkali metal salts of cholesterol phosphate; and mixtures thereof.

8. The cosmetic composition according to claim 1, wherein the non-ionic surfactant with an HLB greater than or equal to about 14 is chosen from esters of polyethylene glycol and stearic acid—PEG-20 Stearate, PEG-32 Stearate, PEG-40 Stearate, PEG-50 Stearate, PEG-100 Stearate, PEG-150 Stearate and mixtures thereof.

9. The cosmetic composition according to claim 1, wherein the non-ionic surfactant with an HLB around about 10 is chosen from polyglycerides, polysorbate 61, polysorbate 65, polysorbate 81, polysorbate 85, polyglyceryl-2 laurate, and mixtures thereof.

10. The cosmetic composition according to claim 1, wherein the non-ionic surfactant with HLB less than or equal to about 6 is chosen from sucrose distearate, sorbitan stearate, octyldodecyl xyloside, glyceryl stearate, and mixtures thereof.

11. The cosmetic composition according to claim 1, wherein the cosmetic composition has an SPF in a range from about 15 to about 55.

12. The cosmetic composition according to claim 1, wherein the cosmetic composition has an SPF in a range from about 55 to about 100.

13. The cosmetic composition according to claim 1, wherein the at least one UV filter is chosen from an organic UV filter and/or a mineral UV filter.

14. The cosmetic composition according to claim 1, wherein the water/oil phase ratio is from about 1.4:1.

15. A cosmetic composition comprising:
a. an aqueous phase;
b. at least one UV filter;
c. an oil phase;
d. an emulsifier system comprising:
i. from about 2.5 to 4.0 wt. % of one non-ionic surfactant with an HLB greater than or equal to about 14 is chosen from esters of polyethylene glycol and stearic acid—PEG-20 Stearate, PEG-32 Stearate, PEG-40 Stearate, PEG-50 Stearate, PEG-100 Stearate, PEG-150 Stearate and mixtures thereof;
ii. from about 0.5 to 1.5 wt. % of one non-ionic surfactant with an HLB around about 10 is chosen from polyglycerides, polysorbate 61, polysorbate 65, polysorbate 81, polysorbate 85, polyglyceryl-2 laurate, and mixtures thereof;
iii. from about 0.25 to 0.75 wt. % of one non-ionic surfactant with an HLB less than or equal to about 6 is chosen from sucrose distearate, sorbitan stearate, octyldodecyl xyloside, glyceryl stearate, and mixtures thereof; and
iv. from about 0.25 to 0.75 wt. % of one anionic surfactant is chosen from alkali metal salts of dicetyl phosphate and of dimyristyl phosphate; alkali metal salts of cholesterol sulphate; alkali metal salts of cholesterol phosphate; and
mixtures thereof.
e. a water/oil phase ratio which is from about 1.3:1.0 to about 2.0:1.0; and
wherein the cosmetic composition is an emulsion with a viscosity less than about 40.0 UD (spindle M1); and
wherein the emulsion produces a mist spray without requiring mixture with propellant;
wherein all percentages by weight are based on the total weight of the cosmetic composition.

16. A method for protecting of inhibiting UV radiation from contacting a keratinous substrate by applying an effective amount of cosmetic composition of claim 1 onto a surface of the keratinous substrate.
